(19) United States
(12) Patent Application Publication
(10) Pub. No.: US 2022/0365075 A1
(43) Pub. Date: Nov. 17, 2022

(54) MULTIDIMENSIONAL MICROFLUIDIC PROTEIN CHARACTERISATION

(71) Applicant: Cambridge Enterprise Limited, Cambridge (GB)

(72) Inventors: Tuomas Pertti Jonathan Knowles, Cambridge, Cambridgeshire (GB); Kadi Liis Saar, Cambridge, Cambridgeshire (GB); Alekszej Morgunov, Cambridge, Cambridgeshire (GB)

(73) Assignee: Cambridge Enterprise Limited, Cambridge (GB)

(21) Appl. No.: 17/764,145

(22) PCT Filed: Sep. 25, 2020
(86) PCT No.: PCT/EP2020/076920

§ 371 Date: Mar. 25, 2022

(30) Foreign Application Priority Data
Sep. 27, 2019 (GB) 1913953.4

(51) Int. Cl. G01N 33/52; G01N 33/68; B01L 3/00; G01N 21/64

(52) U.S. Cl. CPC G01N 33/52; G01N 33/6803; B01L 3/5027; G01N 21/6428; B01L 2300/0816; G16B 40/10

(57) ABSTRACT

The present invention relates to the identification of proteins involving measurement and characterisation of multidimensional aspects of said proteins.

### TECHNICAL FIELD

The present invention relates to the identification of proteins involving measurement and characterisation of multidimensional aspects of said proteins.

### BACKGROUND

The ability to determine the identity of specific proteins is a critical challenge in many areas of cellular and molecular biology as well as in medical diagnostics. The diverse nature of proteins and their central role in a diverse range of biological processes necessitates a requirement for highly specific and sensitive approaches for protein detection and analysis. Indeed, protein detection and characterisation approaches are of fundamental importance for a range of biological and medical research fields, and have provided valuable information for better understanding the onset of disease, including various forms of cancer and neurodegenerative disorders.

At the centre of the discovery of novel protein-based disease biomarkers lies the ability to identify proteins. Protein microarrays are known in the art. By providing a high spatial density array of solid-phase supported affinity reagents, such as antibodies, protein microarrays allow proteins of interest to be selectively captured and subsequently detected through the introduction of a second affinity reagent, which may be fluorescently labelled. As such, in the known art, protein microarray based approaches typically require access to one or more distinct antibodies each targeting a different epitope of a single protein and similarly to any other affinity reagent mediated system. However, their performance is adversely sensitive to undesired cross reactivity events. On a fundamental level, such an affinity-reagent mediated strategy is inherently limited to detecting known targets for which a suitable affinity reagent was consciously included in the library. Thus, disadvantageously, affinity-reagent mediated using consciously selected affinity reagents do not allow for the detection and the discovery of hitherto unknown markers.

Generally, existing protein identification methods can be divided into those that achieve identification through the use of i) affinity reagents (e.g. protein microarrays, as above) and ii) those that identify proteins from their sequence. Mass spectrometry and its various modifications (MS/MS, MALDI) have been at the centre of strategy ii) for decades.

In a typical mass spectrometry experiment, fragments of proteins are formed and separated using techniques such as liquid chromatography before their injection to a mass-spectrometer. This involves intricate and time-consuming preparation steps, and furthermore becomes increasingly challenging in the limit of high molecular weight and low solubility species. Due to these limitations, less than 10% of the mammalian proteome can be accessed through these techniques.

For the analysis of higher molecular weight species, bottom-up sequencing approaches are known, which again usually involve time-consuming proteolysis of a complex mixture of proteins followed by a chromatographic separation of the peptides prior to their sequencing through tandem mass spectrometry (MS/MS). Whether the analysis is performed in a top-down or bottom-up manner, mass-spectrometry generally requires extensive sample preparation steps, often resulting in significant losses, and long experimental analysis time, which is clearly disadvantageous. Moreover, using mass-spectrometry, the presence of less abundant species may be masked by more abundant ones, which limits is usefulness in detecting targets that are present at low concentrations, as is the case for biomarkers during the onset and early stages of diseases. Additionally, the fact that mass-spectrometry necessitates operation in gas-phase presents a challenge in analysis of protein complexes that are held together through transient interactions.

To overcome the drawbacks of mass spectrometry, such as its poor scalability, gas-phase operation and limited dynamic range, Swaminathan et al. (Nature biotechnology, 36(11), p. 1076, 2018) immobilised peptides onto a glass slide and measured their fluorescence through total internal reflection microscopy in consecutive cycles of Edman degradation after selectively labelling Lys and Cys residues. Although avoiding the need for MS, this technique nevertheless similarly relies on obtaining sequence specific information to identify proteins. This includes acquiring full sequences, part sequences or the relative positions of specific amino acids.

Accordingly, there remains a need for new protein detection systems that mitigate or overcome some or all of the disadvantages outlined above.

### SUMMARY

In an aspect of the present invention there is provided a method of characterising an unknown biomolecule using a microfluidic device, said microfluidic device having one or more detection region(s) configured to measure a plurality of global properties of the unknown biomolecule; the method comprising:

introducing a fluid sample containing the unknown biomolecule to the microfluidic device;

using the detection region(s) to measure a plurality of global properties of the unknown biomolecule to obtain a characteristic biomolecule data set; wherein the characteristic biomolecule data set comprises at least three global properties; and

processing the characteristic biomolecule data set to characterise the unknown biomolecule.

In a further aspect of the present invention there is provided a method of characterising an unknown biomolecule using a microfluidic device, said microfluidic device having one or more detection region(s) configured to measure a plurality of global properties of the unknown biomolecule; the method comprising:

introducing a fluid sample containing the unknown biomolecule to the microfluidic device;

using the detection region(s) to measure a plurality of global properties of the unknown biomolecule to obtain a characteristic biomolecule data set; and

processing the characteristic biomolecule data set to characterise the unknown biomolecule.

In a further aspect of the present invention, there is provided a method of characterising an unknown biomolecule using a microfluidic device, said microfluidic device having a plurality of detection regions configured to measure a plurality of global properties of the unknown biomolecule; the method comprising:

introducing a fluid sample containing the unknown biomolecule to the microfluidic device;

using the detection regions to measure a plurality of properties of the unknown biomolecule to obtain a characteristic biomolecule data set;

processing the characteristic biomolecule data set to characterise the unknown biomolecule.

The present invention allows for the first time the ability to identify unknown biomolecules by measuring only global properties. Global properties generally relates to physicochemical properties (physical or chemical property), or residue abundance, of the biomolecule.

The present invention does not rely on obtaining direct sequence specific information. Instead, the identity of the biomolecule (and therefore the sequence) may beneficially be inferred from the measured global properties. This enables rapid identification of unknown biomolecules because it avoids the need to identify protein fragments using mass spectrometry or sequencing individual amino acids to determine the protein sequence. Instead, global properties of the unknown biomolecule can be rapidly obtained to create a characteristic biomolecule data set which can uniquely identify the biomolecule.

Further, the present method obviates the need to perform laborious and time-consuming sample preparation steps which are typically required with mass spectrometry of proteins. In addition, performing mass spectrometry on proteins often results on significant samples losses; this problem is further overcome by the present invention, which can readily perform the analysis on large proteins which may be unsuitable for detection using mass spectrometry.

Consistent with the above advantages, the ability to identity an unknown protein based on measurement of only global properties provides a beneficially high-throughput method.

The present invention also avoids the disadvantages of affinity based identification methods since it does not rely on the pre-selection of suitable affinity reagents. Thus, the present invention is not limited in the proteins it is able to characterise and identify. Said another way, it is not necessary to hypothesise beforehand the targets to be identified and explorative studies across the full landscape can be undertaken as well as screening for specific targets.

In a further aspect of the present invention there is provided a method of characterising an unknown biomolecule, the method comprising:
providing a sample containing an unknown biomolecule to a device having a plurality of detection regions configured to measure a plurality of global properties of the unknown biomolecule; measuring n global properties of the unknown biomolecule to obtain an n-dimensional data set of global property results; and processing the n-dimensional data set to classify the unknown biomolecule.

In a yet further aspect there is provided a microfluidic device for characterising an unknown biomolecule, said microfluidic device comprising a plurality of detection regions configured to measure a plurality of global properties of the unknown biomolecule. Preferably, the device is configured to measure the abundance of amino acid(s) present in the unknown biomolecule, and/or to measure physical properties of the unknown biomolecule. Preferably, the device is configured to measure 3 or more global properties, 4 or more global properties, 5 or more global properties, 6 or more global properties, 7 or more global properties, 8 or more global properties, 9 or more global properties, or 10 or more global properties.

In a yet further aspect there is provided a microfluidic for use in the methods of the present invention, wherein the device comprises two or more separate modules which are configured to operate together to measure the plurality of global properties of the unknown biomolecule and obtain the characteristic biomolecule data set; wherein each module comprises one or mode detection regions, enabling modular selection of the desired global properties to be measured.

### BRIEF DESCRIPTION OF THE FIGURES

Embodiments of the present invention are diagrammatically illustrated, by way of example, in the accompanying drawings, in which:

FIG. 1 shows a schematic of a microfluidic array used to perform identification based on Global protein properties;

FIG. 2a shows a specific example of a fluorescence detection setup used in conjunction with a microfluidic device for protein identification;

FIG. 2b shows a specific example of a microfluidic chip which is capable of measuring global properties of a biomolecule in conjunction with a setup such as in FIG. 2a;

FIG. 3 illustrates a microfluidic top-down identification strategy for identification of proteins from an example set;

FIGS. 4a-4c each show the 1-dimensional data corresponding to the global properties of ratio of a) Tyrosine to Lysine; b) Tryptophan to Lysine; and c) Hydrodynamic radius, for a set of 10 test proteins;

FIG. 4d shows a 3-dimensional plot of the three global properties illustrated in FIGS. 4a-4c, illustrating positions of 10 test proteins;

FIGS. 5a and 5b show, respectively, the probabilistic likelihoods protein identification and misidentification in 3D space; and the confidence levels of identification process using a random forest classifier approach.

FIGS. 6a and 6b show simulated results for, using a set of 13190 proteins, the kernel density estimate for the number of trials (out of 1000) with correct identification out of the target protein when a) the number of global parameters used is two and b) the number of global parameters used is five;

FIGS. 7a and 7b show simulated results for, using a set of 13190 proteins, the kernel density estimate for the number of trials (out of 1000) with correct identification out of the target protein when a) assuming that 95% (2 standard deviations) of the parameters were accurately measured within 10% of the expected value and b) assuming that 68% (1 standard deviation) of the parameters were accurately measured within 10% of the expected range;

FIG. 8 shows a table of correlations between eight global properties, indicating the degree of orthogonality of the parameters with respect to one another;

FIGS. 9a and 9b show simulated results for, using a set of 1000 proteins, the kernel density estimate for the number of proteins out of 1000 correctly identified when a) the hypothetical global properties used are {Lys, Trp, Tyr, M_{w}} and b) when the hypothetical global properties used are {Lys, Cys, Met, charge};

FIG. 10 shows the performance, specifically the median protein identification probability (x-axis) for each combination of parameters up to the total of 8 parameters. Each point corresponds to a different combination of parameters.

FIG. 11a shows a graph with a line of linear regression representing the relationship between measured hydrodynamic radius and molecular weight;

FIG. 11b shows a graph with a line of linear regression representing the relationship between a ratio of fluorescence signals and a ratio of the abundance of Tyrosines and Lysines

FIG. 11c shows a graph with a line of linear regression representing the relationship between a ratio of fluorescence signals and a ratio of the abundance of Tryptophans and Lysines;

FIG. 12a shows a probability map showing estimates for the identification for each of ten unknown protein samples;

FIG. 12b shows a probability map showing estimates for the presence of each of ten proteins in a mixture of said ten proteins;

### DETAILED DESCRIPTION

The invention will now be described with reference to the following non-limiting examples. The following embodiments apply to all aspects of the present invention.

The present invention describes examples, methods and systems of top-down protein identification and characterisation, preferably using microfluidic devices. The method enables rapid identification of a plurality of global properties of an unknown biomolecule which allows for the generation of a unique multidimensional signature that is used to determine the identity of the biomolecule.

The present invention enables identification of unknown biomolecules. The invention is not limited by the type of biomolecules it can identify. Suitable biomolecules include proteins, peptides, modified peptides (including post-translational and chemical labelling modifications), amino acid conjugates of non-proteinaceous nature, non-biological amino acid containing proteins and peptides or amino acid conjugates and the like.

The present invention works by identifying a plurality of global properties of the unknown biomolecule. Examples of the present method which measure such ‘global’ biomolecule properties are in contrast to known affinity-based detection methods, which may have to selectively capture target proteins from a complex mixture. This protein capture typically requires at least one antibody that target only specific epitopes (i.e. specific part of an antigen to which an antibody attaches) on a protein.

Global properties are also distinguished from prior techniques based on mass spectrometry analysis, sequencing, or otherwise determining the specific sequence of amino acids in a protein. Such techniques provide information on the specific sequence to enable detection of the biomolecule. Prior art sequence-based techniques may determine a full sequence, may determine fractions and construct the sequence based on this, and may acquire the relative positions of some amino acids with respect to each other to be able to identify it against a database. In contrast, global properties provide aggregate information on the biomolecule.

By ‘global property’ we mean general physical or chemical properties which, for example, may be readily quantifiable and represented using a single number or label, and preferably measured in a single step. Advantageously, this obviates to the need to pre-select specific antibodies, which in turn require a priori knowledge of protein epitopes. Global properties measure the characteristic of the molecule as a whole.

Global properties include, but are not limited to: measuring the abundance of amino acid(s) present in the unknown biomolecule, for example by means of fluorescence spectroscopy (including the possibility to measure labelled residues, e.g. OPA-labelled Lysine); and measuring physical properties of the unknown biomolecule including size, hydrodynamic radius, Molecular weight (M_{w}) charge/ion binding capacity, iso-electric point (pI), solubility, dipole moment, hydrophobicity, and the like. It can be seen that global properties provide information of a characteristic of a molecule as a whole. Global properties may be measured in separate steps if it is advantageous to do so. Equally, it may be possible to combine information from multiple measurements to yield a new property (for example, determining charge from diffusion coefficient and mobility measurement).

Global properties may be selected on the basis of the degree of correlation or degree of orthogonality. The degree of correlation between two properties is the extent to which one property can be derived from another property. Having low correlation (a high degree of orthogonality) between global parameters increases the ability for the resultant fingerprint to identify a unique biomolecule. There may be low correlation or there may be no correlation between two global properties.

An example of a global property is measuring the percentage abundance of amino acid(s) present in the unknown biomolecule, for example by measuring an intensity of fluorescence emitted at a particular wavelength which is indicative of a particular amino acid.

By measuring select amino acid abundance in an unknown/target biomolecule, it is possible to determine global properties of said biomolecule. Amino acids can be identified by any suitable means. In an embodiment, fluorescence signals for specific amino acids are detected. The amino acid may be unlabelled (i.e. the fluorescence is inherent in and detected from the amino acid itself) or may be labelled with a dye or other marker (such that the conjugated dye or marker is detectable).

Unlabelled amino acid detection: examples of amino acids that can be detected via fluorescence include tryptophan, tyrosine, and phenylalanine. In an embodiment, tryptophan and tyrosine are preferred amino acids to be detected.

Labelled amino acid detection: alternatively, selective amino acids can be conjugated with a suitable dye or marker which is then detected to determine the presence or abundance of amino acid in a biomolecule. Suitable amino acids for labelled detection include lysine, cysteine and methionine. For example, an example of an amino acid suitable for labelled analysis is lysine, which may be labelled with OPA (o-phthaldialdehyde) dye molecules.

In all above examples of detecting amino-acid abundance, in embodiments, a chip or microfluidic device, or other suitable device, may be disposed to measure the fluorescence at more than one wavelength, and/or disposed to provide incident light at different wavelength to induce fluorescence. For example, the device may include a plurality of LEDs having different excitation wavelengths which may be selected based on a particular amino acid which is desired to be measured. As can be seen, either single or multi-wavelength illumination can be advantageously used.

Alternatively, other detection methods are encompassed by the present invention including but not limited to recording scattered and interferometrically scattered signals.

Suitable labels, dye or markers useful for labelled amino acid detection include, but are not limited to OPA (o-phthaldialdehyde) and naphthalene-2,3-dicarboxaldehyde (NDA). Cystine residues can be labelled through maleimide reacting with their thiol groups. Methionine reactions can be performed through redox-activated chemical tagging and are encompassed by the present invention.

In an embodiment, the dye and biomolecule are mixed for a defined period of time to facilitate appropriate conjugation. This may, for example, involve bringing streams together on a microfluidic chip and either let them to mix through passive diffusive mixing or through inducing active mixing (e.g. by generating eddies, by introducing a stirring components such as a magnetic bar). The timescale for this process may be determined by the flow rates of the chemicals in the channels and the channel dimensions and they can be controlled to allow both the mixing process and reaction to occur. Alternatively, in examples, the mixing of the dye and the biomolecule may be performed on-chip (i.e. within channels of a microfluidic device). For example, a microfluidic device may include a labelling channel which allows for such mixing, and enables the biomolecule sample to mix for around, for example, 3 seconds prior to a fluorescent measurement being taken. Thus, only short timescales on the order of a few seconds may be required in order to conjugate the amino acid of interest ready for fluorescence measurement. Different dyes and different amino acids may require different mixing timescales.

In an embodiment, the method uses only unlabelled amino acid detection. In a further embodiment, the method uses only labelled amino acid detection. In an embodiment, the method uses both unlabelled and labelled amino acid detection.

In a further embodiment, a physical measurement of the biomolecule is taken within the device. Examples of suitable physical measurements include M_{w}, Rₕ, charge, pI, dipole moment, solubility or hydrophobicity.

Yet further examples of global properties include, but are not limited to, the following which are listed with their respective references: size (Arosio et al, ACS Nano 10 (1), 333-341); electrophoretic mobility (Herling et al. Applied Physics Letters 102 (18), 184102); electric point (Lapinska et al. Physical Chemistry Chemical Physics 19 (34), 23060-23067); size and charge individually (Herling et al. Physical Chemistry Chemical Physics 17 (18), 12161-12167); size and charge integrated onto a single chip (Saar et al. Microsystems & Nanoengineering 5 (1), 33); ion binding (Herling et al. Biophysical Journal 110 (9), 1957-1966); lysine content (Yates et al. Nature chemistry, 7(10), p. 802); cystine content (Branigan et al. Nature protocols, 8(11), p. 2090); hydrophobicity (Alizadeh-Pasdar et al. J. Agric. Food Chem. 2000, 48 (2), 328-334); IR spectra (Müller, T et al. Lab on a Chip, 14(7), pp. 1315-1319).

Thus, in further examples, the method does not rely on measuring any fluorescent of amino acid to determine global properties, and instead determines a set of global physical properties, for example any one or more from: M_{w}, hydrodynamic radius, isoelectric point, charge, etc.

Collectively, the plurality of global measurements form a multidimensional characterisation of the biomolecule. This multidimensional characterisation creates a signature or fingerprint for the unknown biomolecule. In an embodiment, a four-dimensional signature is obtained by taking four separate measurements of global properties of the biomolecule. In a further embodiment, a five-dimensional signature, a six-dimensional signature, a seven-dimensional signature or more is obtained. As can be seen, the number of dimensions is limited only by the number of individual measurements taken with respect to the unknown biomolecule. Increasing the number of dimensions improves the specificity of the measurement, and additionally allows the present method to better distinguish between a larger set of proteins.

In an embodiment, the specific global measurements are selected in advance to maximise the specificity in relation to a specific target biomolecule. If specific target biomolecules are being analysed, it may be advantageous to select specific global measurements that will best differentiate the target from a set of known biomolecules. Alternatively, if the biomolecule is unknown a wider catch-all panel of measurements could be selected. This enables the user to tune the device to best suit the requirements in a cost effective way by selecting the appropriate number and type of measurements that are undertaken.

In the context of a microfluidic device, all of the global measurements may be determined on a single chip. Alternatively, the device may comprise a number of modular sub-elements to facilitate selection of different properties to be analysed depending on the particular needs. This allows for a flexible system whereby the user can select particular sub-modules that detect the target global property and allows for an efficient and flexible configuration system.

However, in alternative embodiments, the measurements need not be made on a microfluidic device. As such, some or all of the measurements may be made off chip. For example, one or more of the parameters may be determined on equipment that allows acquiring the relevant readings (e.g. a platereader for parameters that involve optical readouts, DLS/zetasizer measurement for hydrodynamic radius and charge).

It is preferable that the global property results are normalised to make the multidimensional characterisation concentration independent. For example, the method may comprise normalizing amino acid proportion data to provide concentration-independent amino acid proportion data. Advantageously, this means that the characteristic biomolecule data will be independent of the concentration of the unknown biomolecule, such that concentration of the unknown biomolecule need not be known a priori. Such normalized data has a reduced dimensionality, such that n data points normalised to give concentration independence with result in an n−1 dimensionality (assuming no degeneracy or underlying linear relationship in the original data points). As such, if measuring amino acid abundance, it is necessary to measure at least two amino acid concentrations to normalise the concentration.

It will nevertheless be understood that certain physical properties, e.g. hydrodynamic radius, are already concentration independent, and thus do not require normalisation. Therefore, normalisation would only be applied on measured global properties which depend on the concentration of the biomolecule in the sample and only when the concentration of the biomolecule is not known, e.g. when the fluorescence signal is indicative of a proportion of amino-acid residue in a protein.

Having generated a multidimensional characterisation signature for the unknown biomolecule, optionally having been normalised to make the multidimensional characterisation concentration independent, the signature is processed to determine the identity of the unknown biomolecule. For example, the method may involve already having at its disposal a pre-determined set of characterisation signatures of a known set of biomolecules. The multidimensional characterisation signature generated for the unknown biomolecule may thus be compared with each of the known signatures in order to determine which (if any) of the known biomolecule is the identity of the unknown one. The comparison may typically involve a quantitative calculation.

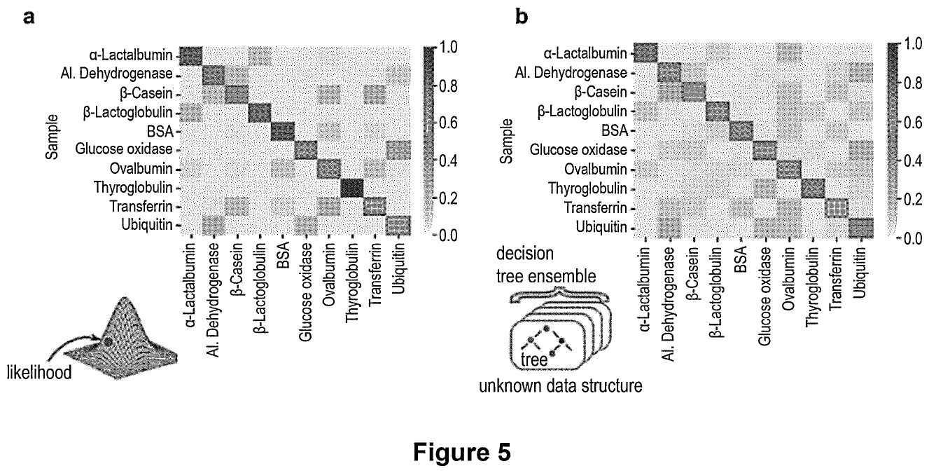

The identity may be determined probabilistically. For example, this comparison may include providing a probability function for each of the known molecules, which accepts as its arguments the measured global parameters of the unknown molecule (the probability functions may have parameters which have been learned/optimised by machine methods as described further below). Each of the probability functions associated with each of the known biomolecules may thus output a likelihood (i.e. a degree of confidence) that it has identified the unknown biomolecule. The biomolecule associated with the function which provides the highest probability may subsequently be selected to identify the unknown biomolecule. This is discussed further below, i.e. in relation to FIG. 5. Alternatively, it might be concluded that the presence of a hitherto unknown biomolecule is identified: merely for example, if none of the probability functions produce a probability of identification higher than a certain threshold (e.g. 50%).

By analysing a plurality of different biomolecules/proteins and examining their identities by a process of mapping their position across a multi-dimensional landscape and using classification and/or machine learning approaches to identify the most likely cluster that each of the samples belonged to, a multi-dimensional signature (for example, a 3D signature in the case of normalised tryptophan, tyrosine and lysine content, and hydrodynamic radius) could be used effectively for uniquely identifying proteins with varying molecular weights. Advantageously, this may be done even when the underlying data distributions are unknown. Thus, examples of the described process suggest that a multidimensional protein characterisation strategy could serve as a powerful probe-free approach for, e.g., an on-chip disease biomarker profiling. Additionally, given the high degree of accuracy with the multidimensional approach can determine the identify of an unknown biomolecule from a set of known biomolecules, the method also provides for the ability to determine the presence of a hitherto unknown protein or biomolecule (for example, in the scenario where the characterisation signature of the unknown biomolecule does not match with any of the signatures of the known biomolecules to a high enough degree of confidence).

Further advantageously, such a strategy requires only microlitre sized sample (given the single-chip microfluidic approach) which may be performed in a very short time frame, requiring minimal preparation steps, and which is applicable to a wide gamut of biomolecule/protein sizes.

In embodiments, measured global properties can be converted into physicochemical parameters, which then enable an unknown biomolecule to be identified based on known physicochemical properties. As such, after experimentally or computationally obtaining a multidimensional signature/fingerprint of an unknown protein, universal relationships can be applied to convert said measured signature into to physicochemical parameters that describe/represent the protein in a universally applicable manner. Advantageously, no a priori knowledge of the unknown biomolecule's unique signature is required in order to identity the biomolecule. Further advantageously, no multidimensional signatures or set of signatures need to be determined or analysed in order for an unknown biomolecule/protein to be identified, since universal physicochemical parameters are determined which are used to directly identify a target/unknown protein or biomolecule.

In embodiments of the present invention, the processing to characterise the unknown biomolecule comprises determining a set of universal parameters from the characteristic biomolecule data set, and determining an identity of the unknown biomolecule based on the set of universal parameters.

In further embodiments, the method comprises determining the identity of the unknown biomolecule comprises determining a likelihood that the set of universal parameters are representative of a set of known universal parameters of any of a set of candidate biomolecules.

In yet further embodiments, the method comprises determining a likelihood that each of a plurality of candidate biomolecules is present in a mixture containing a plurality of unknown biomolecules, wherein the mixture forms part of the fluid sample introduced to the microfluidic device.

In a preferred embodiment, the global properties are identified using a microfluidic device. Microfluidic devices have a number of advantages. They allow only a small amount of sample fluid to be used, and are highly portable devices (compared to potentially cumbersome mass spectrometry machines). Additionally, they can be fabricated quickly and on-demand to suit the requirements of a particular measurement. For example, microfluidic devices may also be modular, allowing for the rapid fabrication of a device for measuring a different set of global properties. The modularity of the microfluidic devices is also advantageous where two sets of measurements of chemically incompatible: in this case, the device may be fabricated such that channels in the device are separated/parallel (as opposed to in series), in order to avoid interference between chemically incompatible regions.

Furthermore, microfluidic devices and associated fluorescent measurement techniques allow for certain measurements to be made in parallel, i.e. two global properties may be measured simultaneously in a single detection chamber. Further advantages include small sample sizes and/or relatively fast measurement timescales.

As such, in embodiments of the invention the microfluidic device may comprise a single detection region configured to measure a plurality of global properties. Said another way, in embodiments of the invention there is a single detection region taking a plurality of measurements. In other embodiments, there are a plurality of detection regions taking a plurality of measurements, with each detection region taking one or more than one measurement.

Microfluidic devices according to the present invention enable the identification of protein samples on a single microfluidic chip, by determining characteristic multidimensional physicochemical signatures of proteins.

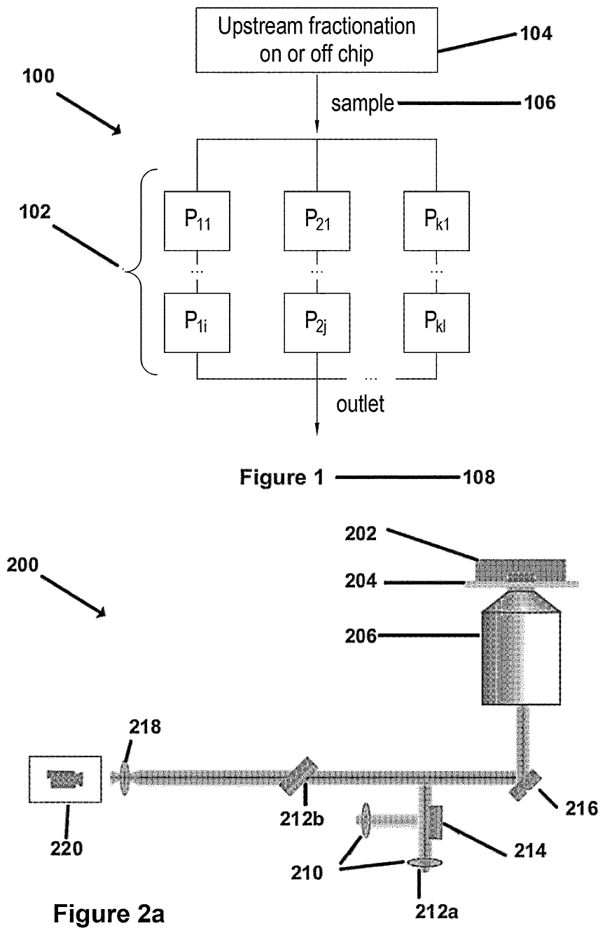

FIG. 1 shows a schematic of an exemplary microfluidic device 100 according to the present invention that can be used to obtain multidimensional signatures. The device involves a purified biomolecule sample entering the chip at an inlet 104 and being transported through a number of individual “analysis units” 102, where the specific parameters are measured either directly or after a labelling step.

Readouts may be of any suitable form but include optical (fluorescence, scattering and the like) or electrical (for example PMT or (s)APD signal). Equally, other types of output are envisaged for example electrical outputs (e.g. current), vibrational or spectral signals.

A purified biomolecule sample would enter the analysis unit where it is transported through a number of processing units also known as property detecting regions (P₁₁-P₁ᵢ, P₂₁-P₂ⱼ, . . . , Pₖ₁-Pₖₗ) where the specific parameters can be measured either directly or after a labelling step. The units are arranged either in series or in parallel and grouped such that the units in series (P₁₁ to P₁ᵢ, P₂₁ to P₂ⱼ, . . . , Pₖ₁ to Pₖₗ) are chemically compatible.

In an embodiment, more than one parameter is obtained from a single unit or region. It is also possible that a specific property is obtained only when the information from a number of units are combined.

For example, a physical measurement may be taken at the same time as identifying an amino acid presence. An example of this would be configuring a region to measure the amount of tryptophan present at the same time as determining the hydrodynamic radius since both measurements can make use of measuring tryptophan fluorescence.

It is also possible that unit(s) are separated into separate modules and configured to allow a user to choose the parameters that are desired to be tested and select the appropriate modules. Via this system, the device can be configured to comprise a plurality of modules, each designed to measure a specific property of the target biomolecule. A user can then mix-and-match from modules to test the required parameters for a given biomolecule. This allows for an efficient manufacturing and design strategy and gives flexibility for users to only test the properties that are of interest to them. As is discussed herein, selection of appropriate properties can increase the sensitivity of the device to particular biomolecules.

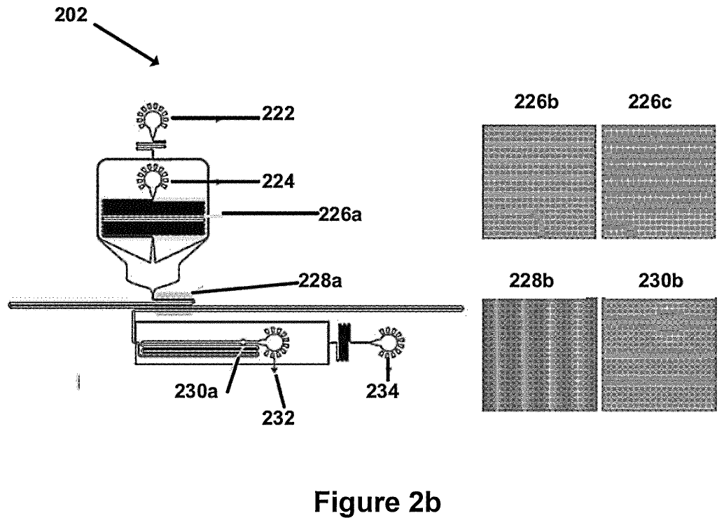

Modules may conduct a single test (i.e. detect a single amino acid). Alternatively, modules may incorporate a number of tests which are deemed complementary, for example for efficiency reasons or because the properties are complementary from a processing perspective. For example, a detection region module in FIG. 2b describes below allows for the simultaneous detection of Tyr and Trp.

FIG. 2a shows an example of a setup 200 for enabling fluorescence detection to be used in conjunction with a microfluidic device for protein identification. The setup include: a microfluidic device/chip 202, a quartz slide 204, an (quartz) objective lens 206, mirrors 214, 215, and 216, and Quartz lenses 210 and 218. The setup 200 further includes LEDs 212b and 212a. In this specific example, LED 212b produces 365 nm wavelength light, and LED 212a produces 280 nm wavelength light. The scene as probed by the LEDs, which are provided to induce fluorescence in the sample contained in the chip 202, may be probed and the global properties ultimately measured using camera 220, which may be an EMCCD (electron multiplying charge coupled device) camera.

FIG. 2b shows an example of a microfluidic device 202. Embodiments of microfluidic devices therefore may contain all, a subset of, or additional features beyond, those in FIG. 2b. It will further be understood that although FIG. 2b shows a device with all modular components in serial, a device may also be fabricated to have parallel channels.

FIG. 2b shows: buffer inlet 222 and a sample inlet 224. FIG. 2b further shows various distinct detection regions 224, 226a-c, 228a-b, and 230a-b in which: 226a is a combined Tyr and Trp detection region, enlarged images illustrating in more detail the channels of the Tyr 226b and Trp 226c detection regions. The sizing region 228a generally is used to determine hydrodynamic radios. The final region is the Lys detection region 230a, shown enlarged in 230b, which may include means for mixing the biomolecule-containing sample with a dye such as OPA dye, to render to Lys detectable by fluorescence. The device 202 also shows a sample outlet 232 and a dye outlet 234.

Generally, the microfluidic devices described herein enable a strategy for obtaining multidimensional physicochemical signatures of biomolecules a single microfluidic chip (or comprising modules thereof). Advantageously, examples of the microfluidic chip design allow obtaining simultaneous readouts indicative of multiple amino acids at once, in addition to physical characteristics of the biomolecule (for example hydrodynamic radius) directly under the native conditions.

An advantage of the present invention is the fact that biomolecules can be investigated in their native state (i.e. without the need to perform laborious or time-consuming sample-preparation steps). In contrast to immobilisation or other techniques, the biomolecule is not in a native state in a suitable liquid. In contrast, the present invention allows measurements of biomolecules in said native state.

Another advantage is that analysis may be carried out under steady analysis conditions and without continuously perturbing the system. An example of this is that the voltage in the device can be fixed and it may not be necessary to continuously adjust voltage.

The method according to the present invention may carry out a fractionalisation step before the biomolecule is introduced into the device. Such an approach will ensure a single biomolecule is tested so as to enable global properties to be assigned to a single biomolecule.

The invention will now be described with reference to the following non-limiting examples.

### Example 1—Microfluidic Device Fabrication

Microfluidic devices were cast using polydimethylsiloxane (PDMS) (Sylgard 184 kit, Dow Corning, USA) from a silicon wafer master imprinted with 50 m high device channels based on standard soft-lithography techniques. The height of the photoresist structures on the master mould were measured by profilometer (DektakXT, UK) to correct for height variations across the master. Carbon black nanopowder (Sigma-Aldrich, UK) was added to the PMDS to minimise undesired auto-fluorescence from the PDMS devices under UV illumination during the measurements. The devices were bonded to a quartz slide (Alfa Aesar, 76.2×25.4×1.0 mm, UK) using plasma treatment (Electronic Diener Femto plasma bonder; 15 seconds at 40% of the full power). The PDMS-glass microfluidic devices were then exposed to an additional extended plasma treatment step (500 seconds at 80% of the full power) to render channel surfaces more hydrophilic with the inlets and outlets blocked with water-filled gel-loading tips immediately after the exposure to prevent the recovery of the hydrophobic channels surfaces.

### Example 2—Microfluidic Device Operation

A UV-LED microscope setup was used in conjunction with the microfluidic device for the multidimensional protein measurements. The schematic of the optical layout is shown in FIG. 2a. The sample was excited using either a 280 nm LED (Thorlabs M280L3, UK) or a 365 nm LED (Thorlabs M365L2, UK) light source with a flip mirror used to switch between the two light sources. The light from either of the LEDs was passed through an aspherical lens of focal length 20 mm to get a collimated output beam. The beam was passed through a dichroic filter cube, which consisted of an excitation filter (Semrock FF01-280/20-25) and a dichroic mirror (Semrock FF310-Di01-25x36). The light reflected by the dichroic mirror was then focussed onto the sample flowing in a microfluidic chip by an infinity corrected UV objective lens (Thorlabs LMU-10X-UVB, UK) of numerical aperture NA=0.25. The emitted fluorescent light from the sample was collected through the same objective and an emission filter (Semrock FF01-357/44-25 for a characteristic tryptophan, FF01-302/10-25 for a characteristic tyrosine and FF01-452/45-25 for a characteristic lysine signal) with an airspaced achromatic doublet lens of focal length 20 mm (Thorlabs ACA254-200-UV) focussing it onto the camera (Rolera EMC2). All the optics used in were made out of fused silica for high transmission in the UV region.

To obtain a multidimensional signature for a sample, the channels of the microfluidic were first filled from the common outlet with phosphate buffer using a glass syringe (Hamilton, 500 L, UK), 27 gauge needle (Neolus Terumo, 25 gauge, 0.5×16 mm, UK), and polyethene tubing (Scientific Laboratory Supplies, inner diameter 0.38 mm, outer diameter 1.09 mm, UK). Gel loading tips filled with the relevant solutions were then inserted into the device inlets (FIG. 2b) and the fluid flow through of the solutions into the channels controlled using neMESYS syringe pumps (Cetoni GmbH, Germany) set to withdraw at a total flow rate of 200 Lh. To increase the accuracy of the diffusional sizing process, the sample inlet was first filled with the auxiliary buffer and a background image of the diffusional sizing area recorded that was subtracted from the sample image before fitting the diffusion profiles. The latter gel loading tip was then carefully changed to another gel loading tip including the protein sample with care taken not to introduce any air bubbles in the process. Finally, in order to account for any potential fluctuations in the power output, the intensities of standard calibration solutions (10 M L-Tryptophan and 10 M 4-methylumbelliferone both in 400 mM potassium borate buffer at pH 9.7) were recorded in a channel adjacent to the top-down identification device itself. The obtained characteristic tryptophan and tyrosine fluorescence values were then normalised by the former of this calibration readings and the lysine value by the latter of the two calibration readings.

Multidimensional Protein Characterisation

FIG. 2b, as described above shows a diagrammatic illustration of a microfluidic setup which may be used to perform initial characterisation and/or detection of a protein. Generally, the same sequence of steps may be followed, an example of which is given below.

A protein sample containing a protein is introduced into a for tryptophan (Trp) or tyrosine (Tyr) detection region from a designated inlet. Fluorescence signals are then obtained for tryptophan and tyrosine content by:
i. exciting the microfluidic chip with a UV wavelength (280 nm) LED (FIG. 2a); and
ii. collecting the emitted fluoresce light with an EMCCD camera using two distinct filters (dichroic mirrors), one each for tryptophan (Trp) or tyrosine (Tyr), that allow the emitted fluorescent light to be measured from either the tryptophan or tyrosine residues, independently of one another.

The sample then exits the Trp and Tyr detection region, and enters a sizing region in which the hydrodynamic radius of the protein within the sample is then measured. This comprises the steps of:
i. surrounding the protein sample with a co-flowing buffer;
ii. monitoring the lateral diffusion protein molecule into an auxiliary carrier medium in space and time, to create a diffusion profile indicative of the mobility of the protein and hence its effective hydrated radius. A camera is used to monitor the diffusion, having a field of view (800 um×1000 um) such that four distinct channels in the sizing region may be observed. The protein-containing channels in the sizing region were imaged using the same 280 nm excitation LED in combination with the tryptophan filter, which produces a stronger emission signal than the tyrosine; and
iii. extracting the diffusion coefficient of the protein from the space/time measurements by fitting the obtained diffusion profiles of the proteins (using micrographs obtained by the camera) to simulated basis functions of known particles (having know radii). Each of the simulated profiles was compared to the measured diffusion profiles to extract the hydrodynamic radius of the protein in the sample.

The sample then enters a third region, which is the Lysine (Lys) detection region, which comprises a labelling channel. The lysine content is thus measured in a third step, in which an on-chip latent labelling strategy is used to conjugate (i.e. form consecutive double bonds along a carbon chain) the lysine residues in each protein, so that they may be labelled with o-phthaldialdehyde (OPA) dye molecules. The characteristic fluorescence intensity from the OPA labelled lysine residues is then measured by switching (by way of a moveable quarts ‘flip mirror’) from the UV-LED light source (280 nm) to the 365 nm LED source, with which the conjugated OPA levels may be probed (since unconjugated OPA molecules shows minimal background fluorescence). Measuring conjugated OPA levels is thus indicative of Lysine content in the protein. The dimensions of the labelling channel were chosen such that the OPA dye and the protein sample would mix for at least around 3 seconds before the measurement was taken. This amount of mixing time advantageously allows quantitative insight into the abundance of Lysine residues in proteins.

Thus, using the above method, it is possible to obtain a four-dimensional signature for each protein sample using a single microfluidic chip and a dual-wavelength (280 nm and 365 nm) excitation system. The four-dimensional signature comprised of four data points is thus unique to the protein, and may be used to identify it. The four data points are: fluorescence intensity of i) Tryptophan, ii) Tyrosine, iii) Lysine contained in the protein, and iv) the hydrodynamic radius (Rh) of the protein. The hydrodynamic radius may be defined as radius of a solute which is equivalent to the radius of a hard sphere that diffuses at the same rate as that solute.

It is then necessary to obtain a multidimensional signature of the protein which is invariant with respect to concentration. Thus, the Tryptophan and Tyrosine fluorescence intensities are normalised using the Lysine intensity. Thus, two fluorescence-intensity ratios are obtained which are concentration independent, and the protein now has a 3-dimensional signature which may be used for either protein classification, or building a classification model.

Protein Classification

Generally, by performing a validation study where the origins of (in this example, ten) test proteins were examined by a process of mapping their position across the multidimensional landscape and subsequently using machine learning approaches to identify the most likely cluster that each of the validation samples belonged to, the described result show that specific proteins can advantageously be identified in this multidimensional landscape at a high accuracy.

Using the microfluidic platform and dual-wavelength (280 nm and 365 nm) excitation system described above in relation to FIGS. 2a and 2b, a set of ten different proteins were analysed.

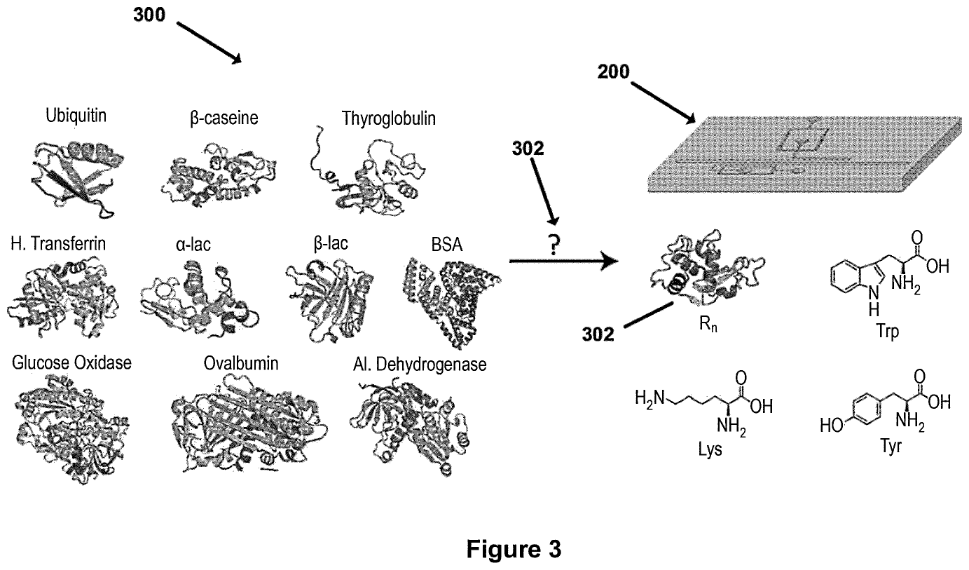

FIG. 3 shows the 10 proteins used in the described proof-of-concept test 300. The measured global parameters of the unknown protein 302 are compared to those of the set of known proteins, in order to identify the unknown protein. This is done using a microfluidic chip 202, which in turn is used to measure global properties: in this case; Hydrodynamic radius (Rₕ), and the residue contents of Trp, Lys, and Tyr.

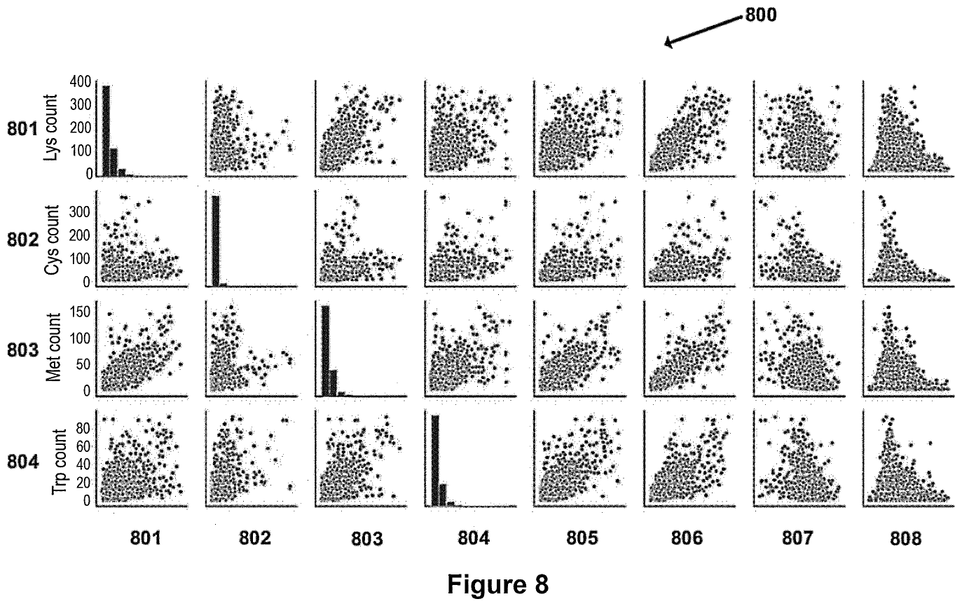

Using the method steps described above, the concentration-independent 3-dimensional signature was obtained for each protein. In this specific example, four repeat measurements were performed on each of the ten proteins, using a different microfluidic device for each repeat. The molecular weight of the set of ten proteins used varied by three orders of magnitude. Nevertheless, the measured Rh (Hydrodynamic radius) values of all the proteins were consistent with known values reported in the literature. Furthermore, the fluorescent intensity of the lysine content scaled linearly with the number of Lysines present (FIG. 8); this is advantageous for using the determined Lysine content to normalise the Trp and Tyr values to gain concentration independent protein parameters.

As mentioned, in order to eliminate concentration dependence, the obtained fluorescence signals measured from the tryptophan and tyrosine imaging channels (FIG. 2b, 226b and 226c) were normalised by the fluorescence signal obtained in the lysine filter (FIG. 2b, 230a). This normalisation reduces the data structure to a three-dimensional signature, and ensures that the obtained values (the values being the obtained Trp/Lys and Tyr/Lys ratios) were independent of the concentration of the protein that was under analysis. As an additional corrective measure, the measured intensities were corrected for fluctuations in the laser power by also measuring the fluorescence intensities of calibration solutions in a neighbouring channel, involving L-tryptophan and 4-methylumbelliferone molecules.

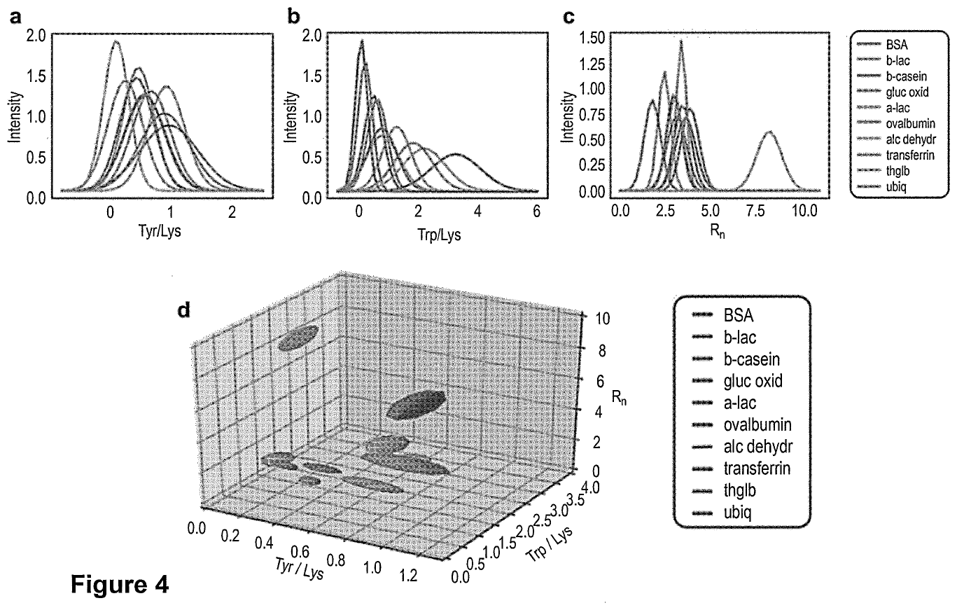

The characteristic space that each of the analysed ten proteins occupies in a three dimensional plot is shown in FIG. 4d, with the 1D projections shown in FIGS. 4a-c. In this specific example, the region in 3D space occupied by each of the ten proteins is defined by an ellipsoid (FIG. 4d). In turn, merely for this specific example, the ellipsoid is defined as having a centre being the average of the four repeat measurement points (i.e. [x, y, z] coordinates), with corresponding radii (along each of the three axes) being the standard deviation of each of the [x, y, z] points for the four repeat measurements. Advantageously, the ten analysed proteins possess varying and distinct physiochemical signatures (as seen in FIG. 4d). In other words, they occupy unique regions in the 3D space according to the three concentration-independent parameters chosen. This illustrates that it is likely that across a three-dimensional landscape each of the proteins possesses a different and unique signature according the 3-dimensional global properties measured for the protein.

Because each of the ten proteins occupy different regions in 3D space, a classification algorithm may be applied to the data in order to train a classification scheme. Thus, the learned classification parameters may then be used to classify an unknown protein sample as being one of the ten sampled proteins. For example, the learned classification parameters may be used to determine probabilistic functions for each of the known proteins, where these functions may take as input arguments a set of global parameters, and output a confidence level that the input arguments relate to a biomolecule of the same identity. Advantageously, this may be done on a single chip, with minimal preparation steps, and within a small time-frame. Two such example methods are described below.

Generally, it will be understood that many example machine learning techniques may be employed (two examples of which are given below) in order to distinguish between a set of characteristic biomolecules signatures in multi-dimensional space. Thus, suitable machine learning methods involving supervised leaning classification algorithms may be used. Essentially, the machine learning process can be viewed as a process of generating decision boundaries between the position in the multidimensional space as defined by the global properties of the biomolecules. For example, positions of 10 molecules in 3D space can be seen in FIG. 4d. Many techniques may be employed for this purpose, including but not limited to: multivariate Gaussian distributions, decision tree ensembles, artificial neural networks (including but not limited to single- or multilayer perceptrons, feedforward neural networks, or deeper architectures including convolutional and residual layers etc.), support vector machines (SVM), Gaussian processes, gradient boosting algorithms, unsupervised clustering methods such as K-means and the like.

Machine Learning (ML) Example 1

Using the full data set of ten classes of proteins in the examples set out in FIGS. 3-5, determined using 4 experimental repeats for each class, leave-one-out cross-validation was used to assess the likelihood that a particular sample is classified as the correct protein. Other suitable techniques include but are not limited to leave-p-out, K-fold, shuffle-and-split, stratified and/or grouped versions of the above.

In one example, multivariate Gaussian distributions were fitted to each of the ten protein classes with the means computed from the four repeats within each class, or from the three remaining repeats for the class from which the validation sample was removed. The covariance matrices were computed by combining the group variance (using either four or three repeats similarly to the means) with the global variance involving the full dataset of 39 data points excluding the validation sample. A weighting factor of 0.9 was used for the group variance and a weighting factor of 0.1 for the global variance to introduce computational stability that would have otherwise raised from the matrix singularity, and, simultaneously, to take advantage of the extra information about the system as the variances in the same dimension between the different classes are likely to be similar. Finally, the likelihood of each of the validation samples belonging to each of the protein classes was calculated by estimating the probability density function of the individual multivariate Gaussians at that point.

For each protein class, the likelihood was averaged across the four experimental repeats and the resulting values were normalised to one. FIG. 5a shows a heat-map of the calculated likelihoods of each protein belonging to a particular class. The numerical probability of the protein being identified as each of the other ten proteins is shown on the vertical axis. That is, the actual protein being measured is shown on the horizontal axis, and the protein it is likely to be identified as on the vertical axis. In this example it was observed that, individually, 7 out of 40 samples were misclassified.

Nevertheless, it can be seen that, on average, proteins are likely to be assigned to the correct class with high confidence. The above probability estimates were obtained by assuming that the errors in the measurements in each dimension were normally distributed. Thus, so the protein classes were approximated by multivariate Gaussian distributions.

ML Example 2

An alternative and potentially improved analysis is detailed below, which comprises an identification/classification strategy that does not make assumptions about the distribution of the points. That is, unlike the Example 1, the distribution of errors in the measurements in each dimension were not assumed to be normally distributed. Thus, a random forest classifier was constructed. As before, leave-one-out cross-validation was used on all 40 samples in conjunction with the random forest classifiers being constructed.

In order to reduce variance, each random forest was trained with 1000 decision trees built using bootstrap, sampled with replacement and with utilising only 2 out of 3 variables at random in each tree. Predictions by the ensemble model were used for performing the classification and predictions by all individual trees in the ensembles were subsequently collected to quantify the confidence of the ensemble model in making the predictions. For each group of four repeat measurements corresponding to the same protein class, the average numbers of trees in the ensemble predicting each target class were taken, and were then normalised to sum to one for each protein.

Finally, a heat-map summarising the results was constructed, showing the actual protein being measured on the vertical axis, and the likelihood of said protein being identified as each of the ten proteins on the horizontal axis (FIG. 5b). The results illustrate that the random forest classifier model predicts the correct class of proteins with yet higher confidence than multivariate Gaussian model (ML Example 1). Moreover, on the individual level, the random forest model misclassified only 4 out of 40 samples, demonstrating a superior performance to the multivariate Gaussian model. This shows that highly accurate identification of proteins is possible, even when no assumptions are made about the underlying distributions of measurement errors or data structure.

This example demonstrates through analysing a set of ten different proteins and examining their identities by a process of mapping their position across the multidimensional landscape and using machine learning approaches to identify the most likely cluster that each of the samples belonged to, that a three-dimensional (or generally an N-dimensional) characterisation signature could be used effectively for uniquely identifying proteins with varying molecular weights even when the underlying data distributions are unknown. These results demonstrate that such a multidimensional protein characterisation strategy serves as a powerful probe-free approach for on-chip disease biomarker profiling using only microlitre sized samples.

Although the above example relates to only 10 proteins and only 4 measured global properties (resulting in an 3-dimensional characterisation signature) it will be understood that the method may readily be scaled to a providing a classification algorithm to a set of thousands or even tens of thousands of proteins, using an arbitrary number of global properties. Depending on the particular selection of global properties selected for use in characterising an unknown biomolecule, it may be necessary to re-train the classification model in order to determine new decision boundaries which relate to the particular selection of global properties. Alternatively, various combinations of global parameters could be used to train a plurality of models to produce a plurality of decision boundaries/classification models a priori, such that, in future, a user may select a desired set of global parameters to measure without the need to re-train the model on those particular parameters.

### Example 3a—Analysis of Method Sensitivity: Number of Parameters Measured

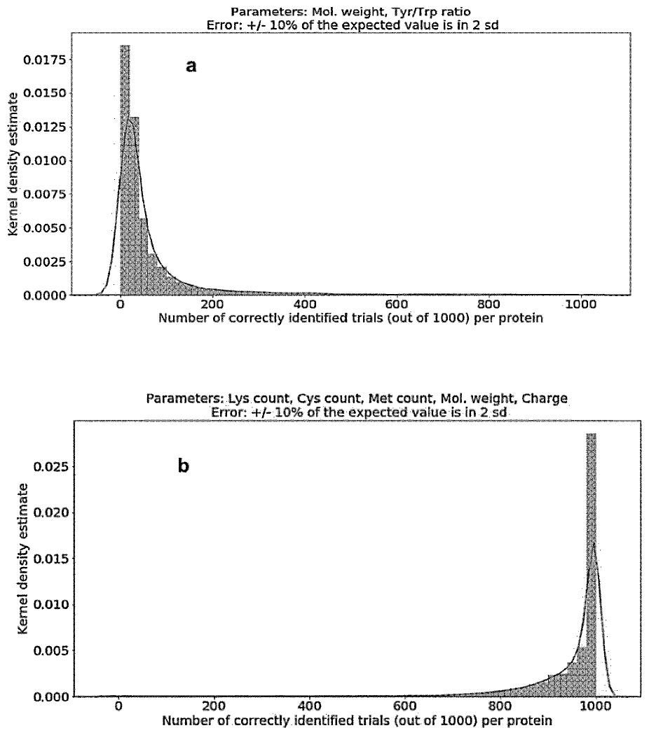

To investigate how the number of measured global parameters can affect identification capability, a setup where identification based on the Tyr/Trp ratio and M_{w} (i.e., fundamentally two global properties) of a protein was simulated. The results are shown in FIG. 6a which shows the kernel density estimate of protein population (y-axis) that gets identified correctly out of 1000 trials (x-axis) when two global parameters are measured. It can be seen that when only two global properties are measured, the median protein has 3.3% chance of being identified correctly, with only 0.27% of proteins having over 90% chance of being identified correctly. This contrasts with a sequencing approach (i.e. a non-global property analysis approach) where measurement of two parameters may be sufficient to identify a protein.

On the other hand, biomolecule identification based on two global parameters may, however, be possible when the targets are to be identified in a background of a smaller number of proteins, for instance when the identification step is performed in a mixture that has been pre-purified, or the analysis is performed after a co-precipitation assay. Nevertheless, it will be understood that, in general, measuring only two global properties provides an unfavourably low identification rate, which might render this method unsuitable in practice.

The results with five global parameters is shown in FIG. 6b, in particular measuring the content of Lys, Cys, Met, M_{W} and charge. By performing the relevant simulations as described above, it was observed that as a result of the increased number of properties measured, the probability of correctly identifying the median protein increased from 3.3% (FIG. 6a) to over 98.8% (FIG. 6b).

This demonstrates the positive impact of using an increasing number of parameters (dimensions) in order to identify biomolecules. Increasing the dimensions can improve selectivity, and greatly improves the degree of confidence with which an unknown protein may be identified as demonstrated with the high identification probability in FIG. 6a.

Further advantageously, measuring a larger number of global properties allows the successful identification of an unknown biomolecule from a much larger data set (a set of thousands of proteins, for example) of known biomolecules, in contrast to the example in FIG. 4 which demonstrates the identification strategy by measuring only 3 global properties with a set of only 10 proteins.

### Example 3b—Analysis of Method Sensitivity: Accuracy of Global Property Measurement

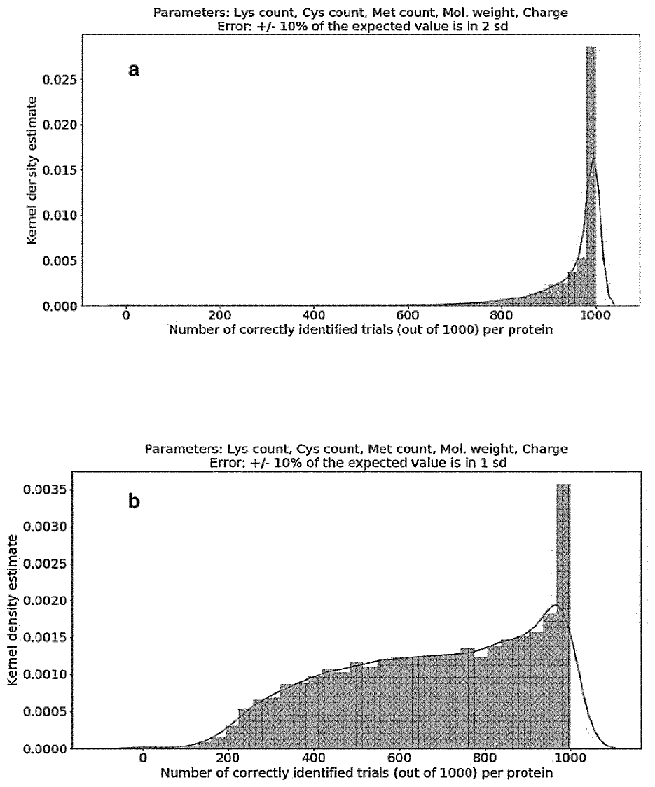

To develop an insight into how the error rate in each of the measurements affects the possibility of achieving accurate identification, the identification capability was compared when the exact same global properties of proteins were measured but to a different level of accuracy. In particular, by using the same 5 measurement parameters as in Example 3a, of Lys, Cys, Met, M_{W} and charge, (i.e. FIG. 6b) but instead of 95% (2 std), assuming that 68% (1 std) of the measurements fell within 10% of the expected value, it can be seen from FIG. 7 that the identification capability of the platform decreased noticeably.

FIG. 7 shows that protein identification capability depends strongly on the accuracy with which each of the global properties is measured. The identification accuracy of a median protein decreases from 98.8% in FIG. 7a to around 70% accuracy in FIG. 7b when 68% (1 std) rather than 95% (2 std) of measurements fell within +/−10% of the expected value.

### Example 3c—Analysis of Method Sensitivity: Parameter Selection

To investigate how the specific types of parameters that are measured can affect the identification capability of the platform, a total of four global parameters were measured.

To identify the optimal combinations of parameters, it was first examined how the measured parameters correlate with each other across the range of analysed proteins. The results of correlations between pairs of seven global parameters are shown in FIG. 8, and it can be seen that parameters such as molecular weight tend to correlate strongly with the content of various amino acids, and therefore this pair of global properties lack orthogonality. In contrast, the charge of molecules correlated only weakly with the content of each of the five amino acids investigated. Therefore, charge and amino acid-content are substantially orthogonal global parameters. In a preferred example, global parameters should be chosen which are all orthogonal or substantially orthogonal to one another, such that more ‘information’ is obtained from the target biomolecule, which in turn will lead to a greater identification accuracy.

FIG. 8 shows the correlation between eight measured global properties (from top to down and left to right: Lys 801, Cys 802, Met 803, Trp 804, Tyr 805, Mw 806, charge 807, and pI 808). This information can be used to evaluate which combination of parameters may give the best performance under the constraint of only a limited number of parameters measured in total. For example, the correlation between Mw 806 and Tyr 805 can be seen to correlate strongly, as seen by the points which are distributed along the x=y axis.

Based on this, it is evaluated whether the identification capability becomes altered when the set of measured parameters is changed. In particular, the combination of {Lys, Trp, Tyr, M_{w}} was hypothesised to perform substantially worse than the combination of {Lys, Cys, Met and charge} due to the much stronger correlations between the parameters in the first set, and the greater orthogonality of parameters in the second set.

It will be understood that the above set of global parameters are merely a subset of the possible global parameters which may be measured. Other global parameters (which may be advantageously orthogonal to those described above) include: iso-electric point; other residues/amino acids including labelled residues; and proportion of the target biomolecule in its pre-fractionation mixture.

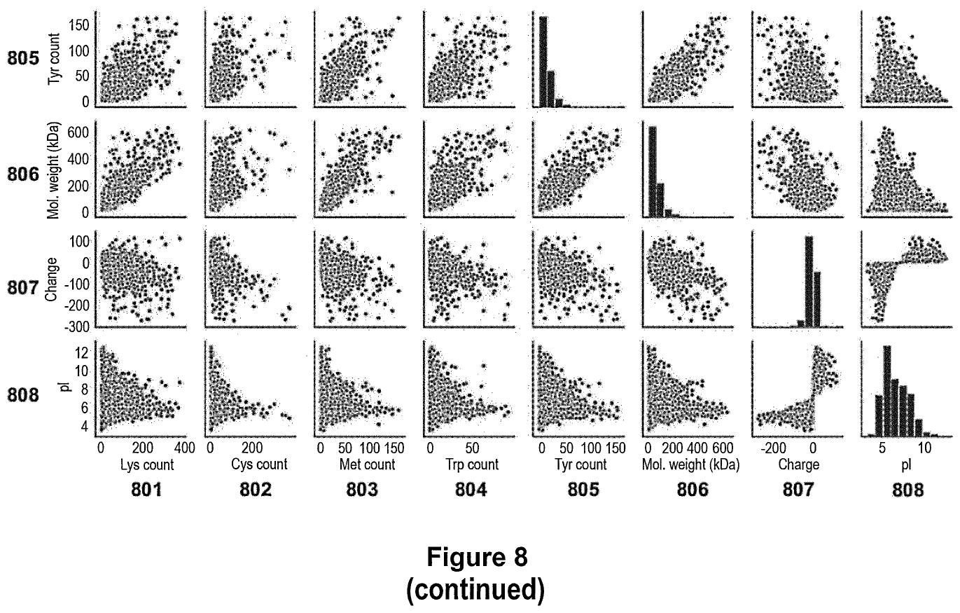

The results are shown in FIG. 9 and confirm a substantial difference in the identification capability between these two sets of four parameters with the average identification capability moving from around 60% in FIG. 9a (corresponding to {Lys, Trp, Tyr, M_{w}}) to close to 93% in FIG. 9b (corresponding to {Lys, Cys, Met and charge}), when more optimally orthogonal parameters are chosen.

This illustrates the advantages in selecting parameters useful to the target biomolecule to maximise selectivity.

Further, a table summarising the identification capability (the median probability that a protein gets identified correctly and the fraction of proteins that have over 90% of probability of being identified correctly) for the data presented in FIGS. 6-7 and 9 is shown below.

| FIG. | median | 90+% identification probability |
|---|---|---|
| 6a | 2.7 | 0.2 |
| 6b & 7a | 98.8 | 84.3 |
| 7b | 70.2 | 22.9 |
| 9a | 60.5 | 16 |
| 9b | 93.3 | 60 |

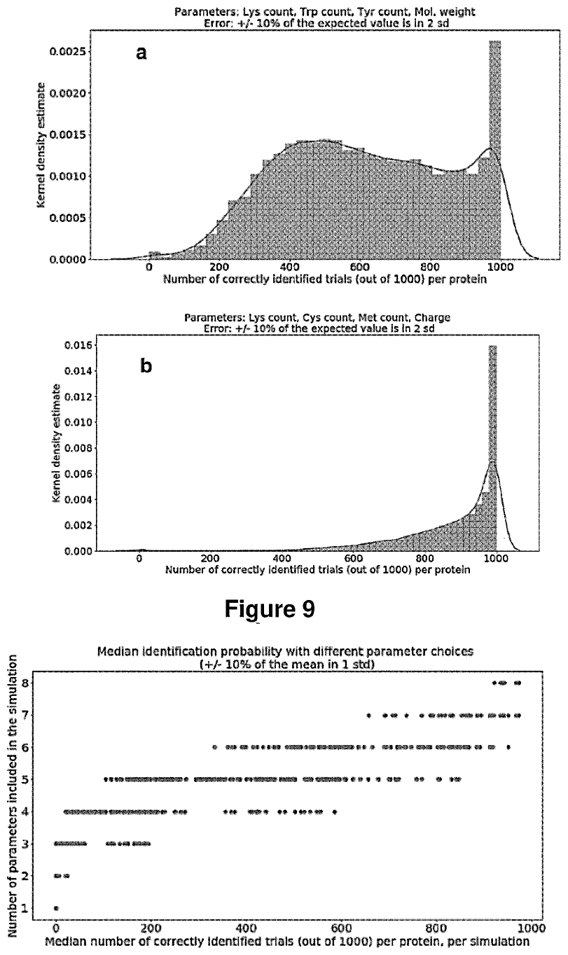

FIG. 10 shows the performance, specifically the median protein identification probability (x-axis) for each combination of parameters up to the total of eight parameters. Each horizontal line corresponds to a different number of combined parameters, and each point corresponds to a different combination of parameters. It can be seen that there is a marked improvement in the median number of correctly identified trials per protein correlating to the number of different global properties. However, and as discussed above in relation to FIG. 8, global properties are not completely independent, and possess a degree of correlation.

Thus, certain combination global properties with a high degree of orthogonality (i.e. a lower degree of correlation) will result in an increased number of correctly identified trials for a given number of parameters. As such, a large variation can be seen (along the horizontal axis) corresponding to varying levels of global property correlation. Nevertheless, this highlights that it is advantageous to choose a set of global properties having as many pairs as possible with a high degree of orthogonality.

Protein Identification

Generally, any of the above methods and techniques may be used to obtain one or more global parameters, which form a multidimensional signature or fingerprint of a protein or biomolecule. We herein describe how these measured global properties can be converted into physicochemical parameters, which then enable an unknown biomolecule to be identified based on known physicochemical properties. In other words, after experimentally or computationally obtaining a multidimensional signature/fingerprint of an unknown protein, universal relationships can be applied to convert said measured signature into to physicochemical parameters that describe/represent the protein in a universally applicable manner.

Advantageously, no a priori knowledge of the unknown biomolecule's unique signature is required in order to identity the biomolecule. Further advantageously, no multidimensional signatures or set of signatures need to be determined or analysed in order for an unknown biomolecule/protein to be identified, since universal physicochemical parameters are determined which are used to directly identify a target/unknown protein or biomolecule.

It will be understood that many possible methods are available for conversion of the measured global parameters (e.g., Hydrodynamic radius, or fluorescence intensities) into physicochemical parameters. For example, mathematical empirical relations can be pre-determined by statistical analysis (for example, regression), i.e. based on a selected group of proteins, where said empirical relation can then be used to calculate physicochemical parameters for an unknown biomolecule based on newly measured global parameters. Furthermore, physicochemical parameters may be established based on literature values, experiments, computationally predicted mathematical relationships, and the like.

### Example 4

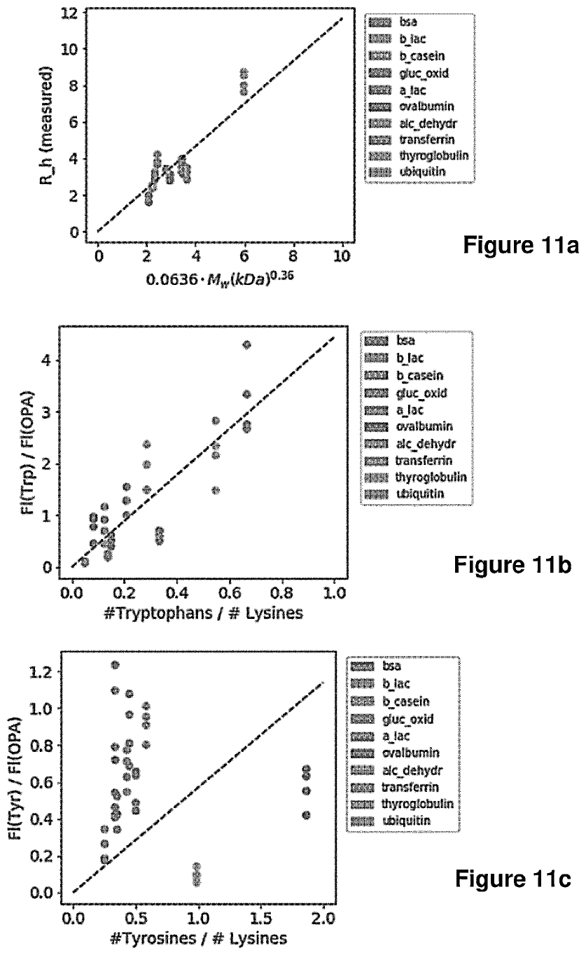

FIG. 11a shows an example in which a test set of 10 proteins have been analysed in order to determine a mathematical relationship between hydrodynamic radius and molecular weight. In this example, a linear regression model has been applied to determine the following relationship:

Rₕ=0.0636×M_{w}(kDa)^{0.36}

The kDa merely represents that the unit used in this example is kilo Dalton, however, any other suitable unit for molecular weight may be used.

FIGS. 11b and 11c show examples where measured ratios of fluorescence signals are plotted, and using linear regression are used to determine a mathematical relationship between a ratio of pairs of fluorescence signals and a ratio of an abundance of amino acids.

Specifically, FIG. 11b shows the ratio of the measured signal of Tryptophan fluorescence to Lysine (tagged with OPA) fluoresce on the y-axis, and the x-axis shows the calculated ratio of the abundance Tryptophan to the abundance of Lysine. The mathematical relationship derived from the line of best fit shown is:

$$\frac{{Fl}_{Trp}}{{Fl}_{OPA}} = 4.44 * \frac{\# Trp}{\# Lys}$$

FIG. 11c shows the ratio of the measured signal of Tyrosine fluorescence to Lysine (tagged with OPA) fluoresce on the y-axis, and the x-axis shows the calculated ratio of the abundance Tyrosine to the abundance of Lysine. The mathematical relationship derived from the line of best fit shown is:

$$\frac{{Fl}_{Tyr}}{{Fl}_{OPA}} = 1.57 * \frac{\# Tyr}{\# Lys}$$

As mentioned above, in some preferable examples, ratios of fluorescence signals are calculated such that the resultant values are independent of sample concentration. However, other examples are possible where individually measured fluorescence signals are converted into an abundance of a single amino acid in a sample/protein. Again, the measured values can be converted to protein/amino acid concentrations or abundances via other suitable methods such as via empirical relationships derived from experimental or literature data.

It will further be appreciated that, in any other the above example in FIGS. 3a to 3c any suitable statistical regression, e.g. non-linear or polynomial regression, model may be used to determine the relationship. Alternatively or additionally, a suitable machine learning method may be applied to learn a mathematical relationship between hydrodynamic radius and molecular weight.

Once the measured fingerprint (the fingerprint comprising a plurality of measurable global parameters) is converted or otherwise recast into more universal physiochemical parameters describing the unknown protein, the identity of the protein may be determined.

The obtained universal parameters may define a physiochemical signature. For example, a score for the physiochemical signature may be calculated in respect of one or more candidate proteins, where that score defines e.g. a closeness or match with said candidate protein. For example, a z-score may be calculated for the obtained physiochemical signature, where the z-score provides an estimated likelihood that the physiochemical signature describes a particular candidate protein. The z score is generally defined as:

$$Z = \frac{x - μ}{σ}$$

where x is the observed/measured value, μ is the mean, and a is the standard deviation for a particular value. Generally, a known physicochemical property of a particular candidate protein may represent the mean, μ, and the measurement noise may define the standard deviation,

For example, the measurement noise may be empirically determined or estimated from a set of test proteins, such those shown in FIGS. 3a to 3c, from a regression line through the data. Thus, classical statistical approaches may be used, or any other measure of e.g. statistical spread can be used to estimate the noise or uncertainty in the experimental measurements.

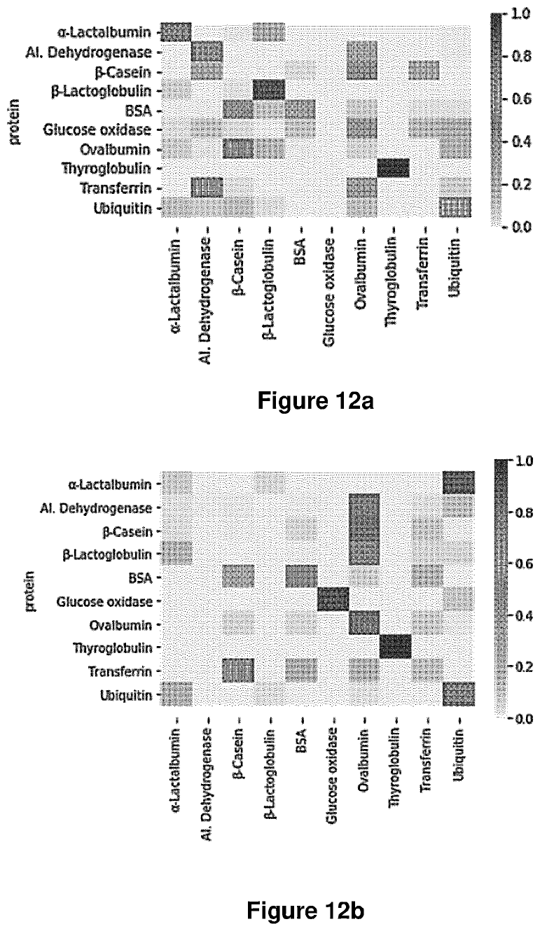

FIG. 12a shows such an example, where unknown proteins have been identified from a set of 10 candidate proteins. Specifically, the probability of each of the 10 measured samples was estimated in respect the likelihood of each measured signature corresponding to a particular candidate protein of the 10 proteins. FIG. 12a shows the results in the form of a heatmap. The actual measured sample is shown in the vertical axis, and the candidate sample against which it is compared, and against which a probability is estimated, is shown on the horizontal axis.

The results show that 5 out 10 proteins were identified correctly (where estimated probabilities are visualised by applying a softmax function). Furthermore, these outlined results were averaged over 4 measurements on each of the 10 proteins, and on the level of an individual experiment, the measured protein was identified correctly in 21 out of the 40 experiments.

Yet further it is possible to show that, in addition to identifying a measured protein using a physicochemical signature obtained from of a measured signature of said protein, it is possible to make predictions of whether a specific protein of interest is present in a set of measured samples.

In this regard, the expected measured fingerprint for a specific protein of interest is evaluated by looking where its amino acid content/abundance crosses a best-fit line (e.g., where such a best-fit line is shown in FIGS. 11a-11c). In other words, that is, an intersection point is determined between a line representing an amino acid value, and the regression line defining the relationship between a measured and physicochemical property. The probability that this point of intersection belongs to any of the experimentally observed clusters may then be determined using any suitable statistical or machine learning approach: for example, a statistical multivariate Gaussian mixture model, or a tree-based classification algorithm such as a random forest classifier, or a gradient boosted decision tree based classifier and the like.

FIG. 12b shows the results of the above described this identification process using the intersection point. Specifically, FIG. 12b shows a probability map describing the probability that a particular protein is present. The y-axis shows the protein whose presence is being tested, i.e. the candidate proteins, where the individual probabilities of each of 10 measured proteins is shown on the x-axis. Furthermore, a function can be applied, e.g. incorporating a threshold, that may convert the estimated probabilities into a binary outcome describing the presence of a particular protein. The function may be some form of a logistic regression function. For example the function may give an outcome of ‘1’ (i.e., indicating that a candidate protein is present) where an estimate probability is equal to or greater than 0.5.

In the experiment described in FIG. 12b, all 10 proteins were present in a mixture. A threshold of a simple logistic regression function was set to 0.5, and 5 of the possible candidate proteins were identified correctly as being present.

It will appreciated that other examples of the experiment in FIG. 12b are possible. Generally, a mixture containing any number of proteins may be provided, and the described approach may be applied to determine the presence in said mixture of any number of candidate proteins based on a determined relationship between measured and universal/physiochemical properties. Furthermore, it will be appreciated that once a relationship has been derived (e.g. using statistical regression as described above, or otherwise), that mathematical relationship may be used to convert measured global properties for any number of unknown proteins, in particular, a protein that had not previously been used to empirically derive the relationship. In this respect, no a priori knowledge of a protein's signature (or indeed any knowledge of the relationship between measured and physicochemical properties for that specific protein) is required in order to make a determination as to the identity of said protein.

In conclusion, the present invention identifies a strategy for obtaining multidimensional physicochemical signatures of individual biomolecules, preferably on a single microfluidic chip. The present invention enables obtaining simultaneous readouts indicative of the multiple global parameters related to the biomolecules, for example tryptophan, tyrosine and lysine content of the biomolecule as well as of their hydrodynamic radius directly under the native conditions.

No doubt many other effective alternatives will occur to the skilled person. It will be understood that the invention is not limited to the described embodiments and encompasses modifications apparent to those skilled in the art and lying within the scope of the claims appended hereto.

1. A method of characterising an unknown biomolecule using a microfluidic device, said microfluidic device having one or more detection region(s) configured to measure a plurality of global properties of the unknown biomolecule; the method comprising:
introducing a fluid sample containing the unknown biomolecule to the microfluidic device;
using the detection region(s) to measure a plurality of global properties of the unknown biomolecule to obtain a characteristic biomolecule data set; wherein the characteristic biomolecule data set comprises at least three global properties; and
processing the characteristic biomolecule data set to characterise the unknown biomolecule.

2. The method of claim 1, comprising a plurality of detection regions.

3. The method of claim 1, wherein the method measures four or more global properties, preferably five, six, seven, eight, nine, or ten or more global properties.

4. The method of claim 1, wherein at least two global properties have a low degree of correlation (a high degree of orthogonality), preferably at least three, four, five, six, seven, eight, nine, or ten global properties have a low degree of correlation.

5. The method according to claim 1, wherein the characteristic biomolecule data set comprises at a least 4 global properties, at least 5 global properties, at least 6 global properties, at least 7 global properties, at least 8 global properties, at least 9 global properties or at least 10 global properties.

6. The method according to claim 1, wherein measured global propert(ies) are normalised to make the results concentration independent.

7.-11. (canceled)

12. The method according to claim 1, wherein a plurality of global properties of the unknown biomolecule are obtained from detecting the unknown biomolecule at multiple wavelengths.

13. The method according to claim 1, wherein the global property includes measuring a physical characteristic of the unknown biomolecule, including but not limited to M_{w}, Rₕ, charge, pI, dipole moment, solubility or hydrophobicity.

14. The method according to claim 1, wherein orthogonal global properties are selected to best differentiate the unknown biomolecule.

15.-16. (canceled)

17. The method as claimed in claim 1, wherein the processing to characterise the unknown biomolecule comprises classifying the characteristic biomolecule data set as being a closest match to one of a set of predetermined characteristic biomolecule data sets, which define the identities of predetermined biomolecules.

18. The method as claimed in claim 17, further comprising:
determining that none of the set of predetermined characteristic biomolecule data sets is a sufficiently close match; and
determining that the unknown biomolecule does not correspond to any biomolecule of the set of predetermined characteristic biomolecule data sets.

19. A method of characterising an unknown biomolecule, the method comprising:
providing a sample containing an unknown biomolecule to a device having one or more detection region(s) configured to measure a plurality of global properties of the unknown biomolecule;
measuring n global properties of the unknown biomolecule to obtain an n-dimensional data set of global property results;
processing the n-dimensional data set to classify the unknown biomolecule.

20. The method as claimed in claim 19, wherein the processing to classify the unknown biomolecule comprises classifying the n-dimensional data set as being a closest match to one of a set of predetermined characteristic biomolecule data sets, which define the identities of a set of known biomolecules.

21. The method as claimed in claim 20, the method further comprising:
obtaining an unprocessed data set comprising a plurality of characteristic biomolecule data sets, one for each known biomolecule; and
applying a classification algorithm to the unprocessed data set, in order to obtain classification data for each of the known biomolecules.

22. The method as claimed in claim 21, wherein the classification algorithm comprises any one of: decision tree ensembles, single or multilayer perceptrons, feedforward neural networks, convolutional neural network, support vector machines, and unsupervised clustering methods such as K-means and the like.

23. The method as claimed in claim 19, wherein at least one of the measured n global properties relates to a concentration or abundance of amino acid, and the method further comprises:
normalising the results of global property measurements to obtain an n−1 dimensional data set being concentration independent.

24. A method of characterising an unknown biomolecule, as claimed in claim 19, wherein the global properties comprise one or more of: physicochemical properties of the biomolecules, and a concentration of amino acid residue in the biomolecule.

25.-26. (canceled)

27. A method as claimed in claim 1, wherein the processing to characterise the unknown biomolecule comprises:
determining a set of universal parameters from the characteristic biomolecule data set, and determining an identity of the unknown biomolecule based on the set of universal parameters.

28. A method as claimed in claim 27, wherein the determining the identity of the unknown biomolecule comprises determining a likelihood that the set of universal parameters are representative of a set of known universal parameters of any of a set of candidate biomolecules.

29. A method as claimed in claim 27, further comprising determining a likelihood that each of a plurality of candidate biomolecules is present in a mixture containing a plurality of unknown biomolecules, wherein the mixture forms part of the fluid sample introduced to the microfluidic device.

30. A method as claimed in claim 14, wherein the selected orthogonal global properties include charge, and amino-acid content.
